(19) United States
(12) Patent Application Publication
(10) Pub. No.: US 2004/0001817 A1
(43) Pub. Date: Jan. 1, 2004

(54) Anti-aging nutritional supplement

(76) Inventor: Vincent C. Giampapa, Montclair, NJ (US)

Correspondence Address: Melvin K. Silverman, Suite 500, 500 West Cypress Creek Road, Fort Lauderdale, FL, 33309, US

(21) Appl. No.: 10/438,247

(22) Filed: May 13, 2003

Related U.S. Application Data
(60) Provisional application No. 60/378,160, filed on May 14, 2002.

(51) Int. Cl. A61K 35/78; A61K 38/43; A61K 31/70; A61K 31/20; A61K 31/198; A61K 33/24

(52) U.S. Cl. 424/94.1; 424/617; 424/725; 514/43; 514/560; 514/23; 514/561

(57) ABSTRACT

An anti-aging nutritional supplement composition includes vitamins, minerals, an inflammatory process support, a blood sugar/insulin support, a botanical antioxidants, a methylating factor, a DNA repair agent, a fat metabolizer, an absorption enhancer, a brain function support, whole foods, a cellular energizer, a nucleotide precursor, amino acids, a fatty acid complex, and digestive enzymes. The composition supplies nutritional supplements necessary for proper glycation, DNA methylation, anti-oxidation, and control of inflammatory processes. The composition and the method of use provide an effective anti-aging treatment by decreasing DNA damage, increasing DNA repair, and improving immune function of human body.

### CROSS REFERENCE TO RELATED APPLICATION

This application is the non-provisional patent application of provisional patent application serial No. 60/378,160 filed May 14, 2002, which is herein incorporated by reference in its entirety.

### BACKGROUND OF THE INVENTION

The instant invention relates to a program of oral supplementation to augment what is termed the cellular soup. The cellular soup constitutes extracellular and intra-cellular fluid which acts respectively to nourish the extracellular matrix, such as tissue and nerves, and the intra-cellular matrix which comprises the inner structure of the cells, this including the cell nucleus and the mitrocondria which are the energy producing elements within every living cell. The cell nucleus is where most genetic functions occur, including the aging process. Accordingly, proper nourishment to the cell nucleus and mitrocondria is an essential aspect of any anti-aging therapy. Within context of the instant invention, the term of cell represents both somatic cell and adult stem cell.

Within the cellular soups are many organic compounds which affect the metabolic process, these including vitamins, minerals, enzymes, amino acids, pre-hormones (known as hormonal precursors), co-factors, antioxidants, anti-inflammatories, anti-glycation agents and DNA methylation control agents.

Each of these agents affects the cellular soup and thereby human metabolism in a particular and distinct fashion. Also, all are necessary to maintain proper metabolic function and to revitalize the basic constituents of the cellular soup to effect cell repair, notably, the cell nucleus and mitochondria, to halt and, to a substantial extent, reverse the aging process. It is therefore essential to maintain, within the cellular soup, high levels of antioxidants, hormonal precursors, RNA, DNA and other of said agents which otherwise decrease with age. Accordingly, by vitalizing, and continually revitalizing, the cellular soup, essential components of cells of the human body will not sense “environmental changes” which they have been genetically taught to associate with the aging process.

Therefore, to the extent that contemporary knowledge has been able to relate the aging process to the status of metabolic functions, the basic building blocks of the cell, namely, its nucleus and mitochondria, can be deceived into believing that no age-related metabolic changes have occurred and, therefore, that there is no reason to change their normal reproductive function of cell replacement.

This is, the aging process is believed to relate to the failure of cell components such as the cell nucleus to continue to replicate otherwise healthy cells. This occurs when damage to essential cell components causes a drop in levels of key chemical agents including specifically those set forth above. These agents will typically diminish as a result of reduction in efficiency, over time, of glands and organs in the human body. Reductions in such key chemical agents are believed to cause damage to the RNA and DNA in each cell nucleus which thereby reduces the ability of the cell to reproduce itself which ultimately brings on the aging process which yet further accelerates a drop in body's production of the key agents set forth above. Accordingly, the essential problem in stopping the aging process is that of maintaining appropriate levels of key metabolic agents, particularly those related to glycation, inflammation, methylation and anti-oxidation, so that the genetic material within each cell will not become damaged or otherwise lose efficiency. Stated otherwise, it is believed that the genetic material of each cell has no way of knowing how old it is, except with reference to the intracellular fluid in which it is immersed, so that, if the extra-cellular fluid is kept youthful, the genetic material within each cell will continue to function in a normal fashion, without decrease in efficiency thereof.

Also of importance in halting the aging process is maintaining the health of the cell surface membrane which is the surface which delineates the extra-from the intra cellular fluid. This cell surface membrane mediates flow of essential metabolic agents between the extra-and intra cellular fluid through the function of receptor sites upon the surface membrane of each cell. There exist numerous types of receptor sites which regulate a wide variety of amino acids, hormonal and anti-oxidant transfer between the extra and intra cellular fluids. Also, the cell surface membrane plays an essential role in the function of so-called ionic pathways between the extra and intra cellular fluid, these facilitating the movement of certain agents, such as minerals, which move electron statically between the extra-and-intra cellular fluid.

The present invention may, accordingly, be viewed in terms of an oral supplementation program designed to restore the integrity of the cellular soup, to neutralize efficiency-impeding end products of metabolism which relate to the aging process, and to supply higher, more youthful levels of antioxidants to better combat otherwise damaging free radicals. With such reduction of free radicals, essential hormonal pre-precursors, including neural hormonal precursors which are essential to the health of brain related glands and the central nervous system, and are the building blocks of DNA, are preserved.

The instant oral therapy program is therefore designed to supplement all cell matrices to ensure provision of necessary hormonal precursors, enzymes, and other necessary building blocks of each cell. This invention more particularly relates to a multi-daily comestible which allows the supply of the equivalent of numerous vitamins, minerals, amino acids, enzymes, supplements, neuro-hormonal precursors, with phytoextracts from plants and precursors for augmenting DNA repair and limiting DNA damage. This invention provides the key vitamins, amino acids, minerals, insulin support agents, methylating factors, fat metabolizers, absorption enhancers, digestive enzymes, and several components of plants and algae which are not obtainable through the average daily diet, and fatty acid complex.

The comestible is taken two to three times daily to maximize the utilization of the vitamins, minerals, amino acids, neurochemical precursors and other nutrients through matching the intake thereof with the needs of the body's natural biorhythm. Studies have shown that the daily administration of such vitamin and mineral supplements result in a much improved level of vitamin and mineral utilization, especially at the cellular level of metabolization. The aid in digestion of food helps the body as the body's own metabolization process decreases with age.

It is thereby to be understood that the present oral supplementation program is designed to maintain a proper level of hormones and hormone precursors to maintain the ability of the body to produce such essential agents as insulin growth factor (IGF), the function of which is to provide growth hormones which, among other functions, stabilize the body against insulin reaction. Growth hormones are further responsible for enabling amino acids and polypeptides with the cell to reach the cell nucleus to thereby enable the cell to properly divide and regenerate itself. IGF also enables polypetides and amino acids to mediate the cellular membrane for the proper nourishment of the cell and helps other agents reach the intracellular fluid. Accordingly, hormone precursors are essential to optimize the body's hormonal response to aging. More particularly, the most significant hormones which relate to aging are believed to be released by the pituitary and thymus glands. As such, through the present oral supplementation program, key hormonal components are strategically enabled, thereby regulating age related hormones and hormone precursors.

A further benefit of the instant oral supplemental program is that of augmenting the key enzymes which aid in human digestion. That is, protecting and assisting the function of the stomach and intestinal system. It has been found that an individual that ingests all necessary metabolic agents will not necessarily achieve the above set forth anti-aging benefits if the digestive tract is unable to efficiently process such agents. Accordingly, an essential aspect of the present system is that of enhancing those enzymes which are essential to human digestion to assure that an individual employing the present system will be able to digest the same in an efficient way so that the anticipated benefits of the system can be realized.

In addition, it has been found that, with age, the efficiency of the intestine or gut will deteriorate. Accordingly, the ability to utilize nutrients in an efficient manner drops as a function of age, thereby accelerating the aging process. Thus, no program of nutrient and anti-free radical supplementation can be successful unless those enzymes which aid digestion and which otherwise protect the intestinal tract are a part of such a system.

The prior art, as is known to the inventor, consists of individual and multiple vitamin and supplements which only give partial benefits relative to those of the instant invention. Existing vitamin or mineral supplements, or other prior art known to the inventor, do not address the problems addressed by the instant invention. That is, other vitamin or mineral supplement systems do not provide needed supplements in a manner synchronous with the biorhythmic demands which dictate the timing of biologic need for specific doses of supplements which are required for specific times in the cell repair cycle.

The prior art in the present area is represented by such publications as Food, Nutrition, and Diet Therapy—a Textbook of Nutritional Care, by Krause and Mahan published by W. B. Saunders Co., 1984. This paper and others of its type properly recognize the effect of aging upon metabolic functions but fail to recognize that the metabolic changes also affect the aging process. Further, the prior art as represented in the above does not recognize the effect of the daily bio-cycle upon the capacity of vital organs, such as the pituitary, thalamus and pancreas, in producing hormones as a function of the time of the day when hormone related agents are ingested. Also, the prior art does not correlate efficiency of the digestive tract to capacity of an individual to efficiently metabolize essential nutrients including vitamins, minerals, amino acids, and neuro-hormonal precursors in order to form the essential hormones of the endocrine system. Accordingly, prior art therapies do no include digestive tract enzymes in anti-aging regimen.

Several biomarkers have been reported that can independently indicate DNA damage accumulation. These are interleukins 1a and 1b which are proinflammatory cytokines produced by inflammatory cells responding to oxidative stress signals; 8-hydroxy guanine DNA adducts in lymphocyte DNA; and plasma/serum protein thiols which are a surrogate indicator of endogeneous oxidative stress production by estimating the conversion of thiols to disulfides which in turn can indicate critical DNA repair dysfunction such as with poly ADP-ribose polymerase (PARP). Thiol status of serum/plasma proteins have already been shown to estimate poly ADP-ribose polymerase (DNA repair) activity and longevity of mammals (Pero et al Biochimie 77: 385-393, 1995; Pero et al J. Anti-Aging Med. 3(3): 241-249, 2000). Therefore, plasma thiols surrogate estimate DNA repair capacity, and consequently the potential level of DNA damage remaining.

DNA damage as the source of cellular mutations and one of the primary causes of chronic diseases in man is now one of the oldest and best substantiated medical hypotheses (Cross et al Ann. Intern. Med. 107: 526-545, 1987; Bohr et al Toxicol. Lett. 102103: 47-52, 1998). Oxidative stress relating to the metabolic rate of oxygen consumption, dietary factors and environmental exposures has been identified as the major environmental factor capable of predisposing individuals to elevated levels of DNA damage (Cross et al Ann. Intern. Med. 107: 526-545, 1987; Cerutti Science 227: 375-381, 1985). Oxidative stress can be genetically inherited such as familial polyposis and ulcerative colitis, or acquired such as HIV infections and oxygen-radical generated genotoxic exposures from diet. Moreover, the contribution of oxidative stress to the down regulation of DNA repair has also been documented (Bohr et al Toxicol. Lett. 102/103: 47-52, 1998; Lieber et al Am. J. Path. 153(5): 1323-1332, 1998). Together all the data from the literature provide amble evidence for estimating individual sensitivity to oxidative stress as primary evidence for successful anti-aging therapeutic intervention.

On another aspect, aqueous extract of Cat's Claw has been shown recently having the ability to promote the repair of damaged cells in the body. Cat's Claw has been used historically as a medicinal plant source by the Native Indians of South America for over 2000 years. Cat's claw is a vine which is shredded and traditionally prepared as a tea that can be taken either hot or cold as a supplement in the treatment of many human disorders including inflammations, cancer, and infections. Because of its historical medicinal use, Cat's claw products have been offered commercially in the USA and abroad for hundreds of years in preparations of pulverized plant parts, and water and ethanol extractions. Cat's Claw has been well established as a safe herbal supplement.

U.S. Pat. Nos. 6,039,949 and 6,238,675 (to Pero) teach a special aqueous extract of Cat's Claw, commercially named C-MED-100®. Different from other Cat's Claw products, C-MED-100® is 100% water soluble, and therefore 100% bio-available for absorption by the body. In laboratory tests, C-MED-100® demonstrated no toxicity when dosed in rodents 100 times greater than the recommended dose. Extensive scientific studies have shown that C-MED-100® possesses the ability to promote the repair of damaged cells in the body, and also has a profound effect on cells that were damaged beyond repair and reproducing in large numbers. C-MED-100® showed an ability to cause these cells to cease their dangerously uncontrolled reproduction.

It would be desirable if the effect of C-MED-100® can be utilized together with other supplement to achieve an enhanced anti-aging efficiency.

### SUMMARY OF THE INVENTION

In one embodiment, the present invention provides an anti-aging nutritional supplement composition which comprises vitamins, minerals, an inflammatory process support, a blood sugar/insulin support, a botanical antioxidants, a methylating factor, a DNA repair agent, a fat metabolizer, an absorption enhancer, a brain function support, whole foods, a cellular energizer, a nucleotide precursor, amino acids, a fatty acid complex, and digestive enzymes. The composition supplies nutritional supplements necessary for proper glycation, DNA methylation, anti-oxidation, and control of inflammatory processes.

In a further embodiment, the present invention provides an anti-aging treatment method. The method includes orally administering three doses of the instant composition, with one in the morning, one at midday and one at night respectively. Furthermore, the concentrations of various components of the composition are different among the three doses for the purpose to best support human body bio-cycle's need.

It is an object of the invention to provide a system to biochemically supplement all necessary vitamins, minerals and other nutrients to maintain proper cell, metabolism and body function in an individual comestible taken three times daily.

It is another object to supply the body with key vitamins, minerals and other nutrients to aid the body in metabolization of food complexes, to thereby assist in cellular regeneration and immune system repair, and to augment DNA repair thereby decreasing age-related DNA damage.

It is a further object of the invention to reduce the effect of aging by increasing the digestive and metabolic capabilities of the body.

It is another object to provide a comprehensive vitamin, mineral and nutrient supplement system congruent with natural bio-rhythms to thereby maximize metabolization, proper hormonal formation, release, and utilization of the supplements of such a system.

It is a further object to provide appropriate acidity to both the extracellular and intracellular matrices.

The above and yet other objects and advantages of the present invention will become apparent from the hereinafter set forth Detailed Description of the Invention.

### BRIEF DESCRIPTION OF THE DRAWINGS

FIG. 1 is a diagram of the aging equation to which the present invention is directed.

FIG. 2 is a diagram showing the consequences of insufficient DNA repair.

FIG. 3 is a diagram showing an overall anti-aging treatment strategy of which the present nutritional supplement is a part.

FIG. 4 comprises a summary of age management therapy goals.

FIG. 5 shows the test results of DNA damage estimated by the presence of (8-OH) guanine adducts per 109 DNA bases in peripheral lymphocyte DNA isolated from blood samples of the subjects collected before (baseline), 4 weeks after supplement (treatment) and after 2 weeks more washout (non-treatment).

FIG. 6 shows the redox balance, as a surrogate estimate of DNA repair, analyzed in plasma samples of the subjects as nmoles cysteine in 0-80% ammonium sulfate precipitated protein before (baseline), after 4 weeks supplement (treatment), and after 2 weeks more washout (non-treatment).

FIG. 7 shows the test results of interleukin 1a determined in plasma samples of the subjects as pg/ml before (baseline), after 4 weeks supplement (treatment), and after 2 weeks more washout (non-treatment).

FIG. 8 shows the test results of interleukin 1b determined in plasma samples of the subjects as pg/ml before (baseline), after 4 weeks supplement (treatment), and after 2 weeks more washout (non-treatment).

### DETAILED DESCRIPTION OF THE INVENTION

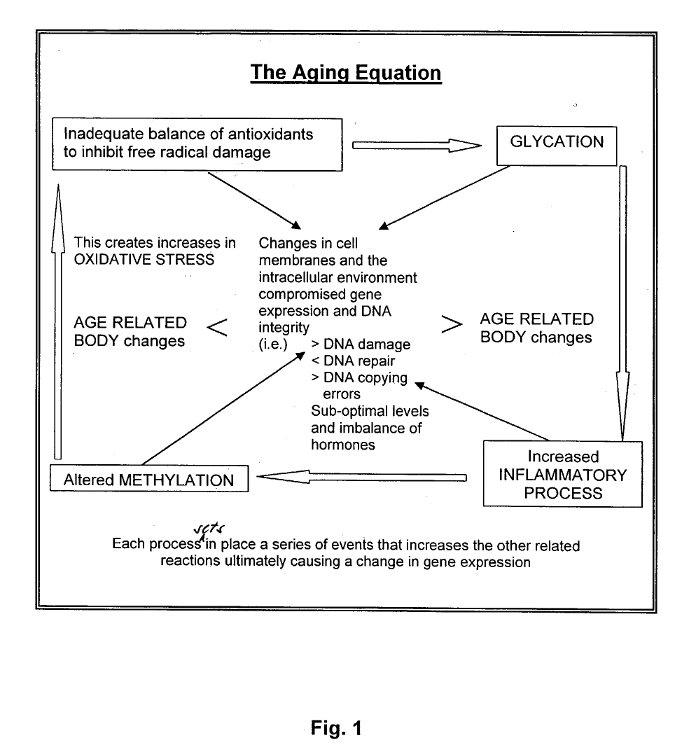

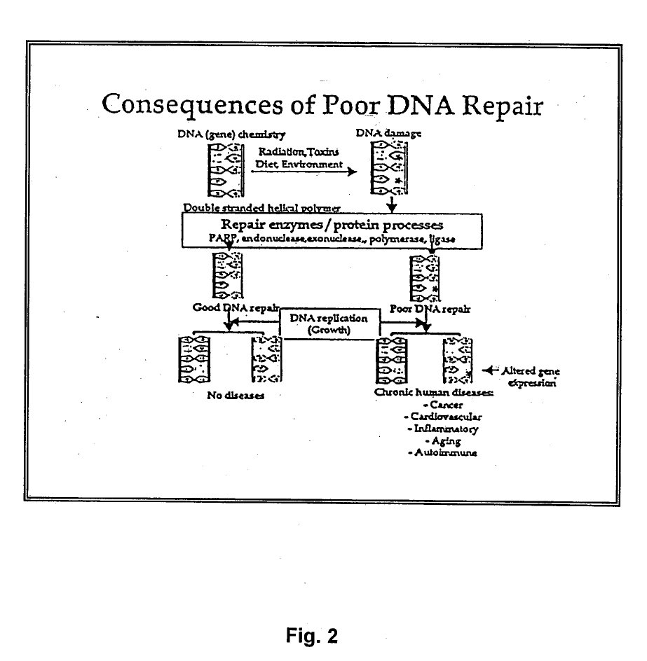

The fundamental process of aging can be viewed as an aging equation encoded within our 46 chromosomes. This genetic code controls the aging equation. The key processes of aging can be summarized with the understanding that the primary focus of treatment should center around control of glycation, inflammatory processes, and oxidative stress in the form of decreasing free radicals to avoid DNA damage, and improving DNA repair, as well as improving the process of methylation of DNA, as illustrated in FIGS. 1 to 2. These are the cornerstones for a successful clinical treatment of the aging process, in a documentable improvement and reversal of key biomarkers of aging and improvement in age-related changes in the body and face.

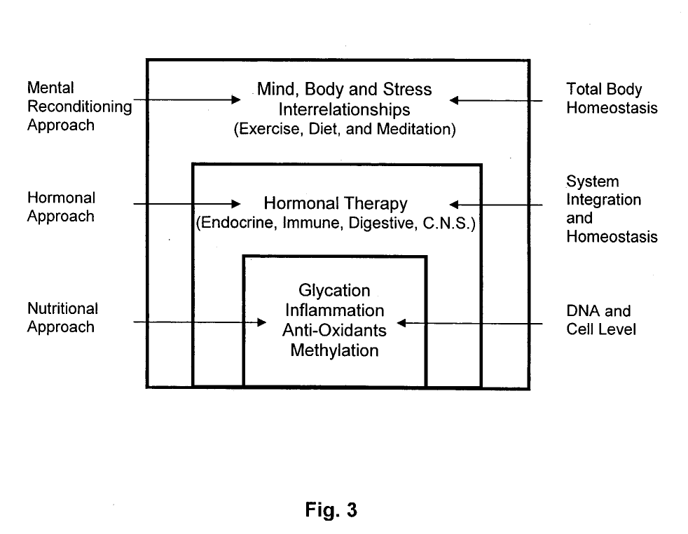

As shown in FIG. 3, control of glycation, inflammation and methylation, and antioxidation are the building blocks over which other anti-aging treatments are laid upon. In this illustration, through a nutritional and pharmaceutical approach, we can markedly affect gene expression and DNA at an intracellular level. As we positively impact upon these processes, they directly affect hormonal levels, which help restore the balance and function of the endocrine system, immune system, digestive system, and the central nervous system. It is at this level that these systems begin to self-integrate among each other.

A homeostasis effect is accomplished that is similar to what is present in a youthful healthy individual. Built upon these improvements are the observable total body changes that occur with exercise, diet and mind-body techniques, as illustrated in FIG. 3. It is for this reason that these key fundamental processes at the DNA and cellular levels are then an essential focus in anti-aging medicine and age management programs. Therein, if one can successfully improve glycation, inflammation, oxidation and methylation processes, to a significant level, the other overlying components are positively affected as well.

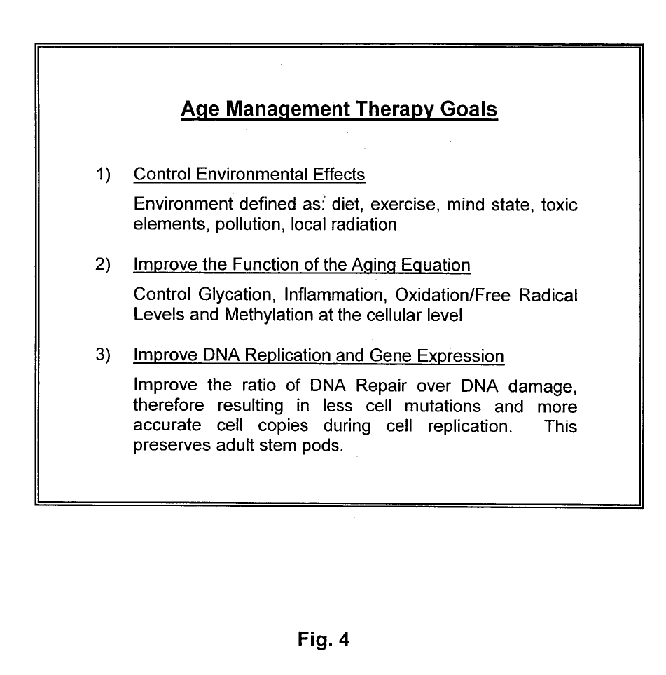

For the cosmetic surgeon, this approach forms the basis of an age management program, as illustrated in FIG. 4. As noted therein, every one of these changes is directly related to changes in both the face and body and is based on the scientific fact that each one of these four key components affects DNA function. This, ultimately, results in the physiological changes that occur as one ages. This process also affects our stem cell pool production, which impact virtually every organ in our body.

In evaluating an overall treatment program, it is essential to focus on the following clinical goals, summarized as: (1) Decreasing DNA damage, (2) Increasing DNA repair, (3) Augmenting immune function, and (4) Optimizing genetic expression.

Over the last few years, it has been documented that neutraceuticals (vitamins, minerals, phytochemicals, enzyme complexes), as well as prescription drugs, directly influence gene expression. That is, they may down-regulate certain genes and up-regulate other genes. If this is done in the correct fashion, they can alter cell signaling, and directly affect hormonal levels. Hormones are ultimately responsible for changes in proteins and other growth factors that occur, which directly impact tissue regeneration and bodily changes on the physical level.

Glycation

For improving control of glycation, supplements that will improve insulin sensitivity are recommended. These include alpha lipoic acid, especially in the time-release form “Glucotize,” along with vanadium, chromium, zinc, taurine, fenugreek, and bitter gourd (Medical Research Institute of California). The prescription use of Metformin or Glucophage in micro-doses at 125 mgs two times a day with lunch and dinner is an ideal way to control blood sugar elevation and loss of insulin receptor sensitivity. Low glycemic index diets and exercise are all essential parts of this process as well. An ideal macronutrient ratio can directly affect the key hormones of aging. Micronutrients such as the essential amino acids, lysine, ornithine, citrulline, curcumin, Vitamin E, and phytonutrients, and co-enzyme Q-10 are also key compounds in improving glycation and dysglycemia.

Antioxidants

An anti-oxidant program is essential to help combat the constant free radical production one experiences as part of life. Anti-oxidant supplements containing vitamin A, vitamin C, vitamin E, selenium and zinc, in moderate doses, and a host of compounds from vegetable derived foods are essential to control free radical levels. Free radical levels are directly related to NF-kappa-B stimulation, or inhibition, which is one of the more important compounds in determining the rate of DNA repair. The rate and quantity of DNA damage have been shown to be directly related to elevated free radical levels at both the nuclear and cell membrane level.

Deprenyl has been shown in experimental studies to improve the intrinsic anti-oxidant levels that we normally produce on our own. These compounds, superoxide dismutase catalase, and glutathione peroxidase, are produced at the intracellular level and also at the level of the mitochondria, which is the site of maximum free radical production and free radical damage.

Inflammation

Control of the inflammatory process centers around the implementation of a low allergy diet supplemented with digestive enzymes. These enzymes break down the protein peptides to amino acids and aid in absorption. Gastrointestinal support with such compounds, such as lactobacillus and amino acids like I-glutamine, along with said digestive enzymes, markedly decrease the inflammatory processes that occur at the cellular level.

Appropriate amounts of ratios of omega-6 and omega-3 essential fatty acids are one of the key ways of regulating the inflammatory pathways within the cell. Niacinamide, glucosamine sulfate, as well as folic acid, are all essential natural approaches Lipoic acid, boswelic acid, turmeric, gymnenma ginger, and bitter gourd also directly impact the inflammatory process as well as act as natural Cox 2 inhibitors.

Inhibiting NF-kappa-B is also important to slow aging at the cellular level and especially in the stem cell pool reserves. C-Med 100™, a specially processed aqueous extract of cat's claw, has been shown to inhibit both NF-kappa-B and TNF-alpha, the key intracellular inflammatory compounds.

Methylation

Methylation is the process where certain genes are “turned on” and other genes are “turned off.” It can be improved by additional supplements of Vitamin B-6, B-12, folic acid and other methyl donors (i.e., betine, trymethylglycine), and Sam-E (S-adenosyl methionine) Decreasing cortisol levels also improves methylation. The easiest way to do this is by augmenting DHEA levels, which decrease body fat levels, as well as agerelated brain cell death associated with related memory impairment.

Energy

Another essential component to an anti-aging treatment is to improve mitochondrial oxidative functions, or mitochondrial production of ATP which is the essential energy intermediate from which all cellular processes, as well as DNA repair are accomplished. Keeping ATP production optimal is an essential component to improve aging efficiency. Improving mitochondrial oxidative function can be accomplished with additional levels of lipoic acid, n-acetylcysteine, naicinamide, co-enzyme Q-10, lipoic acid, I-carnitine, and microhydrin, a micro-clustered silicon compound that acts as a strong hydrogen donor. Also, important at this level, are the additional compounds such as taurine, Vitamin E, and glutathione.

pH Levels

Improving water quality is an essential component as well in age management. Since our cells are 98% water, and water is the medium in which all of these biochemical processes occur, it is essential to have the appropriate quantity and quality of water. Healthy water should be more alkaline, which helps to balance the intracellular and extra cellular pH levels. An improvement in water surface tension and wetting ability, which are supplied by compounds like silica and micro cluster compounds described previously, will improve the cells' hydration capability and membrane permeability, therefore allowing cellular toxins to flow out of the cell and key essential micronutrients to flow into the cell along the key electrolytes.

Immunity

Improving immune function can also be accomplished with components from yeast cell extracts known as beta-1,3-glucans. The herbal compounds echinacea, golden seal and astragalus have also been documented to improve T-cell function and immune function. The above-mentioned C-Med 100® is also a potent white blood cell stimulant, thus improving DNA repair within white blood cells, as described in detail hereinafter.

C-Med-100® is a specially processed aqueous extract of Cat's Claw, Uncaria tomentosa, manufactured by Laboratorio Centroflora (Sao Paulo, Brazil) and distributed in North America by AF Nutraceuticals (Morristown, N.J.). It is a water soluble extract ultra-filtrated to remove high molecular weight toxic conjugates (>10,000 MW), containing 8-10% carboxy alkyl esters (CAE) as active ingredients. It is essentially free of oxindole alkaloids (<0.05%).

The present invention provides complex anti-aging nutritional supplement compositions in suitable pharmaceutical forms, such as tablets, capsules, and caplets, which can be administrated orally one to three times a day. To support body's bio-cycle, it is preferred to take the supplement composition three times daily, more particularly, in the morning, at midday, and at night. Furthermore, for the optimal effect, the supplement composition of the present invention is formulated into three different compositions, which are specifically used in the morning, “AM Formula”; at midday, “MD Formula”; and at night, “PM Formula”, respectively.

One example of the specific compositions of the three formulae is as follows:

The AM Formula comprises in four caplets:

(1) Vitamins

Vitamin A (as retinyl palmitate and 85% as beta-carotene from D. salina algae) 3,500 IU

Vitamin C (as ascorbyl palmitate and ascorbic acid) 200 mg

Vitamin D (as cholecalciferol) 67 IU

Vitamin E (as d-alpha tocopheryl succinate and with mixed tocopherols) 100

Thiamin (as thiamin HCl) 10 mg

Riboflavin (as riboflavin and riboflavin 5-phosphate) 1 mg

Niacin (as niacinamide and niacin) 125 mg

Vitamin B6 (as pyridoxine HCl and pyridoxal 5-phosphate) 25 mg

Folate (as folic acid) 100 mcg

Vitamin B12 (as cyanocobalamin) 150 mcg

Biotin 100 mcg

Pantothenic acid (as D calcium pantothenate) 25 mg

(2) Minerals

Calcium (as calcium carbonate and calcium citrate) 500 mg

Iodine (as potassium iodide and from kelp) 50 mcg

Zinc (as zinc glycinate) 4 mg

Selenium (as selenomethionine) 60 mcg

Copper (as copper lysinate) 0.4 mg

Manganese (as manganese gluconate) 0.4 mg

Chromium (as chronium polyicotinate) 100 mcg

Molybdenum (as sodium molybdate) 20 mcg

(3) Blood Sugar/insulin Support—Blend

Vanadium (as vanadyl sulfate) 50 mcg

Fenugreek seed, Alpha-lipoic acid and Coenzyme Q-10 75 mg

(4) Botanical Antioxidants

Green tea leaf extract (40% catechin and polyphenols) 100 mg

Tumeric rhizome extract (95% curcuminoids) 50 mg

Ginkgo biloba leaf extract (24% ginkgo flavonglycosides, 6% sesquiterpene lactones) 100 mg

(5) Methylating Factors

Betaine HCl 8 mg

Sulfur (from MSM—methylsulfonylmethane) 2.5 mg

(6) DNA Repair Agent

C-Med-100® (extract of Uncaria tomentosa, standardized to 8% Carboxy alkyl esters manufactured by Laboratorio Centroflora (Sao Paulo, Brazil) 150 mg

(7) Fat Metabolizers

L-Carnitine-L-tartrate 100 mg

Acetyl L-carnitine HCl 75 mg

(8) Absorption Enhancers

Phosphatidylcholine (from soy lecithin) 50 mg

(9) Brain Function Support

dimethylaminoethanol (DMAE) bitartrate 50 mg

(10) Whole Foods—Blend 250 mg

Blue-green algae, Spirulina algae and Green barley grass (aerial parts)

(11) Cellular Energizers

Cordyceps sinensis fungus extract (1% cordycepic acid) 25 mg

Royal Jelly 3× (5% 10-HDA) 25 mg

(12) Nucleotides-Precursors for Gene Expression

Ribonucleic acid (from yeast) 100 mg

(13) Amino Acids—Blend 275 mg

Taurine 50 mg

N-Acetyl-L-cysteine 25 mg

(14) Fatty Acid Complex

Omega III complex (7.5% eicosapentaenoic acid and docosahexaenoic acid from fish body oil) 300 mg

(15) Probiotic Complex 100 million CFU

Lactobacillus acidophilus, Lactobacillus plantarum, Bifidobacterium bifidum and Lactobacillus casei

(16) Digestive Enzymes—Blend 1,760 Units

Amylase, Neutral protease, Lactase, Lipase and Cellulase

The AM Formula further comprises microcrystalline cellulose, croscarmellose sodium, stearic acid, calcium silicate, magnesium stearate, silica, and film coat (hydroxypropyl methylcellulose, hydroxypropyl cellulose and polyethylene glycol).

The MD Formula comprises in four caplets:

(1) Vitamins

Vitamin A (as retinyl palmitate and 85% as beta-carotene 2,800 IU

Vitamin C (as ascorbic acid and ascorbyl palmitate) 400 mg

Vitamin D (as cholecalciferol) 56 IU

Vitamin E (as d-alpha tocopheryl succinate and from mixed natural tocopherols) 80 IU

Thiamin (as thiamin HCl) 12 mg

Riboflavin (as riboflavin and riboflavin 5-phosphate) 8 mg

Niacin (as niacinamide and niacin) 200 mg

Vitamin B6 (as pyridoxine HCl and pyridoxal 5-phosphate) 16 mg

Folate (as folic acid) 160 mcg

Vitamin B12 (as cyanocobalamin) 240 mcg

Biotin 80 mcg

Pantothenic acid (as D-calcium pantothenate) 40 mg

(2) Minerals

Calcium (as calcium carbonate) 400 mg

Iodine (as potassium iodide and from kelp) 24 mcg

Zinc (as zinc glycinate) 3.2 mg

Selenium (as selenomethionine) 48 mcg

Copper (as copper lysinate) 0.3 mg

Manganese (as manganese gluconate) 0.3 mg

Chromium (as chronium polyicotinate) 80 mcg

Molybdenum (as sodium molybdate) 16 mcg

(3) Blood Sugar/Insulin Support—Blend

Vanadium (as vanadyl sulfate) 40 mcg

Fenugreek seed, Alpha-lipoic acid and Coenzyme Q-10 55 mg

(4) Botanical Antioxidants

Ascorbigen 8 mg

Cruciferous vegetable concentrate: broccoli, kale, radish (2% glucosinolates) 80 mg

Grape skin extract (37% total polyphenols) 40 mg

Lutein (from marigold flower extract) 1.6 mg

(5) Methylating Factors

Betaine HCl 6.4 mg

Sulfur (from MSM—methylsulfonylmethane) 2 mg

(6) DNA Repair Agent

C-Med-100® (extract of Uncaria tomentosa, standardized to 8% Carboxy alkyl esters manufactured by Laboratorio Centroflora (Sao Paulo, Brazil) 100 mg

(7) Fat Metabolizers

L-Carnitine-L-tartrate 100 mg

Acetyl L-carnitine HCl 60 mg

(8) Absorption Enhancers

Phosphatidylcholine (from soy lecithin) 40 mg

(9) Whole Foods—Blend 100 mg

Blue-green algae, Spirulina algae and Green barley grass (aerial parts)

(10) Cellular Energizers

Cordyceps sinensis fungus extract (1% cordycepic acid) 20 mg

Royal Jelly 3× (5% 10-HDA) 20 mg

(11) Nucleotides-Precursors For Gene Expression

Ribonucleic acid (from yeast) 80 mg

(12) Amino Acids—Blend 310 mg

L-Glutamine, Taurine, L-Tyrosine, and N-Acetyl-L-cysteine

(13) Fatty Acid Complex 320 mg

Omega III complex (7.5% eicosapentaenoic acid and docosahexaenoic acid from fish body oil) 300 mg

(14) Probiotic Complex 80 million CFU

Lactobacillus acidophilus, Lactobacillus plantarum, Bifidobacterium bifidum and Lactobacillus casei

(15) Digestive Enzymes—Blend 1,408 Units

Amylase, Neutral protease, Lactase, Lipase and Cellulase

The MD formula further comprises microcrystalline cellulose, croscarmellose sodium, stearic acid, calcium silicate, magnesium stearate, silica, and film coat (hydroxypropyl methylcellulose, hydroxypropyl cellulose, and polyethylene glycol).

The PM Formula comprises in four caplets:

(1) Vitamins

Vitamin A (as retinyl palmitate and 85% as beta-carotene from D. salina algae) 2,300 IU

Vitamin C (as ascorbic acid and ascorbyl palmitate) 165 mg

Vitamin D (as cholecalciferol) 44 IU

Vitamin E (as d-alpha tocopheryl succinate and with mixed natural tocopherols) 65 IU

Vitamin K (as phytonadione) 6.5 mcg

Thiamin (as thiamin HCl) 0.65 mg

Riboflavin (as riboflavin and riboflavin 5-phosphate) 10 mg

Niacin (as niacinamide and niacin) 140 mg

Vitamin B6 (as pyridoxine HCl and pyridoxal 5-phosphate) 3 mg

Folate (as folic acid) 65 mcg

Vitamin B12 (as cyanocobalamin) 200 mcg

Biotin 65 mcg

Pantothenic acid (as D-calcium pantothenate) 32 mg

(2) Minerals

Magnesium (as magnesium oxide and magnesium glycinate) 265 mg

Zinc (as zinc glycinate) 2.5 mg

Selenium (as selenomethionine) 40 mcg

Copper (as copper lysinate) 0.2 mg

Manganese (as manganese gluconate) 0.2 mg

Chromium (as chronium polyicotinate) 65 mcg

Molybdenum (as sodium molybdate) 12 mcg

(3) Blood Sugar/Insulin Support—Blend

Vanadium (as vanadyl sulfate) 32 mcg

Fenugreek seed, Alpha-lipoic acid and Coenzyme Q-10 50.5 mg

(4) Botanical Antioxidants

Tomato lycopene extract (20% lycopene) 16 mg

Rosemary 4:1 extract (aerial parts) 6.5 mg

Pycnogenol (pine tree bark extract) 3.3 mg

(5) Methylating Factors

Betaine (as betaine-HCl) 5 mg

Sulfur (from MSM—methylsulfonylmethane) 1.5 mg

(6) DNA Repair Agent

C-Med-100® (extract of Uncaria tomentosa, standardized to 8% Carboxy alkyl esters manufactured by Laboratorio Centroflora (Sao Paulo, Brazil) 100 mg

(7) Fat Metabolizer

L-Carnitine-L-tartrate 65 mg

(8) Absorption Enhancers

Phosphatidylcholine (from soy lecithin) 32 mg

(9) Brain Function Support

Inositol 100 mg

Kava Kava root standardized extract (30% kavalactones) 65 mg

(10) Cellular Energizers

Cordyceps sinensis fungus extract (1% cordycepic acid) 16.5 mg

(11) Nucleotides-Precursors for Gene Expression

Ribonucleic acid (from yeast) 65 mg

(12) Amino Acids—Blend of Essential and Non-Essential Amino Acids and Fatty Acids 813.5 mg

L-Arginine (as L-arginine HCl), Omega-III fish body oil complex (4.5% EPA and 3% DHA), L-Omithine (as L-omithine HCl), Taurine and N-Acetyl-L-cysteine

(13) Probiotic Complex 65 Million CFU

Lactobacillus acidophilus, Lactobacillus plantarum, Bifidobacterium bifidum and Lactobacillus casei

(14) Digestive Enzymes—Blend 1,169 Units

Amylase, Neutral protease, Lactase, and Lipase and Cellulase

The PM Formula further comprises: dicalcium phosphate, microcrystalline cellulose, croscarmellose sodium, stearic acid, silica, magnesium stearate, silica, and film coat (hydroxypropyl methylcellulose, hydroxypropyl cellulose, and polyethylene glycol).

As shown above, many of the individual components within a specific functional group are the same among the three compositions, however, the amount of the individual component can be different because of different needs of body's bio-cycle at different time. For example, the amounts of vitamins are different among AM, MD and PM formulae. Moreover, the active components within a specific functional group can also be different, for example the components of botanical antioxidants are different among AM, MD and PM formulae. It is also noted that the amount of individual component or the blend can vary in the formulae, depending on the source, purity and potency of the component.

Additionally, although the above-described formulae are in a three dose form, the formulae can also be administered once or twice a day, which provides relatively lower overall potency. It has also been found when a person takes three doses some components, such as whole foods, brain function support and probiotic complex, can be provided in only one, or two of the doses. Moreover, the C-MED-100® can be provided in the AM and PM formulae only with a slightly higher concentration, such as 175 mg.

A double blind placebo controlled study on the above-described anti-aging nutritional supplement was performed by the Giampapa Institute for Anti-Aging Medicine, Montclair, N.J., in conjunction with the University of Lund, Sweden, and Immunoscience Labs, Beverly Hills, Calif. The study was to confirm the effectiveness of the broad spectrum anit-aging nutritional supplement to reduce oxidant-induced DNA damage in humans, and to establish C-Med-100®, the ability of the DNA repair enhancing nutritional supplement, to further enhance the effectiveness of the anti-aging nutritional therapy.

10 female and 9 male volunteers having age from 35 to 55 years old were involved in the study. The volunteers were asymptomatic and were randomly assigned to Group 1 or Group 2 Group 2 subjects administered the above-described three compositions, hereinafter referred as AM Formula 2, MD Formula 2 and PM Formula 2 and together as Formulae 2, with a dosage of 4 caplets each time. Group 1 subjects administered the same three compositions, except that the compositions did not contain C-Med-100®, hereinafter referred as AM Formula 1, MD Formula 1 and PM Formula 1 and together as Formulae 1, with a dosage of 4 caplets each time. The subjects were supplemented daily for 4 weeks, then supplement discontinued for 2 weeks (referred as a washout period), and the two groups crossed-over for an additional 4 weeks. Heparinized peripheral blood samples were collected from the subjects before supplement, 4 weeks after supplement, 2 weeks after washout, and finally 4 weeks after the crossed-over supplement for analysis of plasma interleukin 1a, plasma interleukin 1b, 8-hydroxy guanine DNA adducts (hereinafter referred as 8-OH adducts) and plasma thiols in the 0-80% ammonium sulfate precipitated protein fraction.

Exclusion criteria for this study were more than two (8-OH) adducts per 109 cells before supplement, any significant effects on more than two biomarkers after cross-over of 4 weeks supplement, or failure to comply with protocol through to the end of the 2 weeks washout period sampling. Applying these criteria, the cross-over data was not included because there still remained significant effects of supplement on the biomarkers even after 2 weeks washout, and only 3 males and 8 females completed the protocol with more than two (8-OH) adducts per 109 cells. The subjects were encouraged not to change their diet during the supplement period, nor to take any other supplements other than those prescribed by the doctor for this study.

It is further noted that among the subjects no apparent acute or chronic diseases were reported by either the subjects or the attending physician before or during this study. There were also no side effects or symptoms observed or recorded that could be attributed to either supplement or to the washout period for the subjects being evaluated. Physical examination and interviews conducted by the attending physician also revealed no apparent medical changes in signs or symptoms.

Four biomarkers that can independently indicate DNA damage accumulation were used in this study. These were: (i and ii) Interleukins 1a and 1b which are proinflammatory cytokines produced by inflammatory cells responding to oxidative stress signals; (iii) (8-OH) guanine adducts in lymphocyte DNA; and (iv) plasma/serum protein thiols which are a surrogate indicator of endogeneous oxidative stress production by estimating the conversion of thiols to disulfides which in turn can indicate critical DNA repair dysfunction such as with poly ADP-ribose polymerase (PARP).

For interleukins 1a and 1b analyses, the biomarkers were determined in plasma by immunoblotting technology known in the art, and performed by Immunosciences Lab, Inc., Beverly Hills, Calif. (8-OH) adducts was also determined by Immunosciences Lab, Inc on lymphocyte DNA isolated from the subjects' blood samples.

The plasma/serum protein thiol test was performed following the procedure described by Pero et al (Pero et al, J. Anti-Aging Med. 3(3): 241-249), and was performed by a Campamed reference lab (Department of Cell and Molecular Biology, University of Lund, Lund, Sweden). The test procedure used is described as follows: 5 μl of the serum samples were diluted 1:15 with saline (75 μl), and then 5 μl aliquots were assayed in duplicate in 96-well microtiter plates. 200 μl of BCA/Cu reagent were added per well (10 ml bicinchoninic acid (BCA) supplied as Sigma B-9643+0.2 ml 4% CuSO₄×5H₂O, made-up fresh before use), and then incubated 30 min at 37° C. The blue color absorbance was read at 540 nm in a spectrophotometer. For background calculations, 5 μl saline/well replaced the protein fractions in the reaction mixture. Note that the serum samples are assayed as a 15-fold dilution compared to the thiol determination, which in turn represents only 1/40 (i.e., 5 μl out of 200 μl serum) of the thiol sample size (1/15×1/40=1/600 dilution factor correction). 5 μl aliquots of dialyzed sera were quantified for protein by comparison to a standard curve of 0-10 μg bovine albumin (in 5 μl dissolved in saline). Alternatively non-dialyzed sera could also be quantified for protein by producing a standard curve of serum supplemented with 0-10 μg bovine albumin and analyzed in 5 μl aliquots. Both techniques required calculation to the standardized sample volume of 200 μl serum as described above.

The statistical analyses of the test results were performed as paired t-test comparisons of sample group means using SPSS software package (from SPSS, Inc.) before, after 4 weeks supplement and after two more weeks washout of both Group 1 and Group 2 subjects.

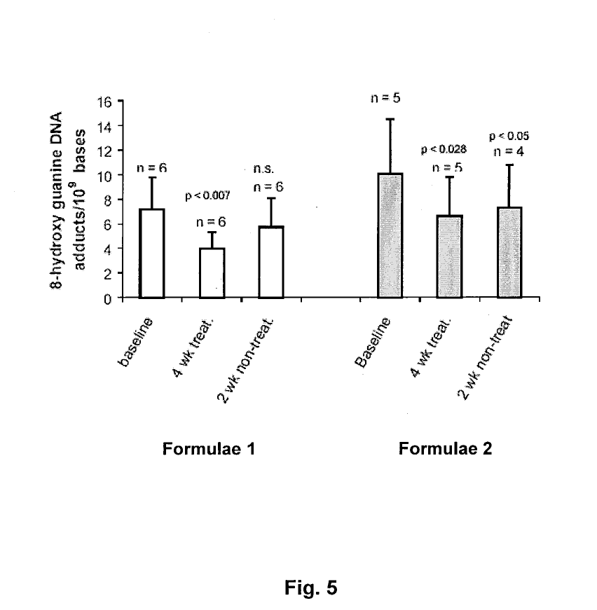

FIG. 5 shows the test results of (8-OH) guanine DNA adducts. The direct measure of DNA damage in peripheral lymphocytes was used in this study as the benchmark biochemical test to evaluate the efficacy of anti-aging nutritional supplement. The data shown in FIG. 5 demonstrated that both Formulae 1 and Formulae 2 were very effective at reducing DNA damage in lymphocytes exposed daily in vivo to the supplements for 4 weeks. More importantly, the (8-OH) adducts/109 nucleotide bases in DNA after Formulae 2 supplementation remained significantly reduced even after 2 weeks washout. Since Formulae 1 contains all ingredients of Formulae 2 except C-Med-100®, the more persistent reduction in DNA damage achieved by Formulae 2 can be directly attributed to the presence of C-Med-100® in the composition and synergetic effect of C-Med-100® with other supplements.

As described previously, plasma/serum thiols are a good estimate of endogenous oxidative stress in vivo as they reflect reduction/oxidation (redox) balance throughout the body due to peripheral circulation (i.e., exposure) of blood to all tissues. Because amino acids such as cysteine can easily react with oxidative radicals converting thiols to disulfides, then the relative balance of thiols to disulfide forms can be estimated colormetrically to indicate the redox balance in serum. The estimates made for this study were carried out on 0-80% ammonium sulfate precipitated proteins in plasma, in order to avoid simple modulation from dietary components such as cysteine, uric acid, vitamin C, carotenoids, glutathione, etc., and thus allowed the focus on redox balance measurement to be concentrated on potential signal transducting proteins in plasma.

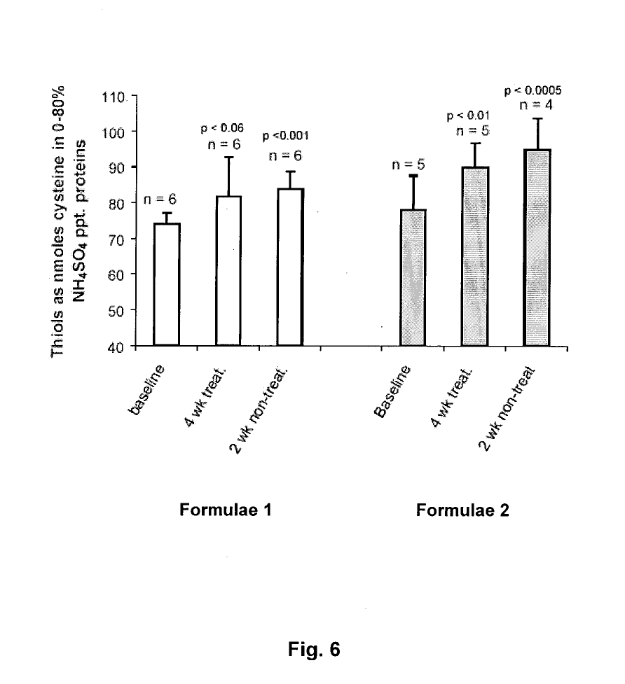

FIG. 6 shows the test results (paired t-test analysis) of plasma/serum thiols as a surrogate estimate of DNA repair, which were analyzed in plasma samples of the subjects as nano-moles cysteine in 0-80% ammonium sulfate precipitated protein before (baseline), after 4 weeks supplement (treatment), and after 2 weeks more washout (non-treatment).

As shown, the samples from both groups of subjects which administered either Formulae 1 or Formulae 2 for 4 weeks showed a concomitant elevation in plasma/serum thiol status. This clearly indicated that these dietary interventions were successful in reducing the endogenous oxidative stress levels of the supplemented subjects. This result was strongly supported by the (8-OH) adduct data presented in FIG. 5, because increased DNA repair reflected by increased thiol status in plasma would predict less DNA damage. Moreover, the magnitude of the thiol increase and the significance level supported that the C-Med-100® containing Formulae 2 was more effective at enhancing DNA repair and thereby resisting DNA damage accumulation.

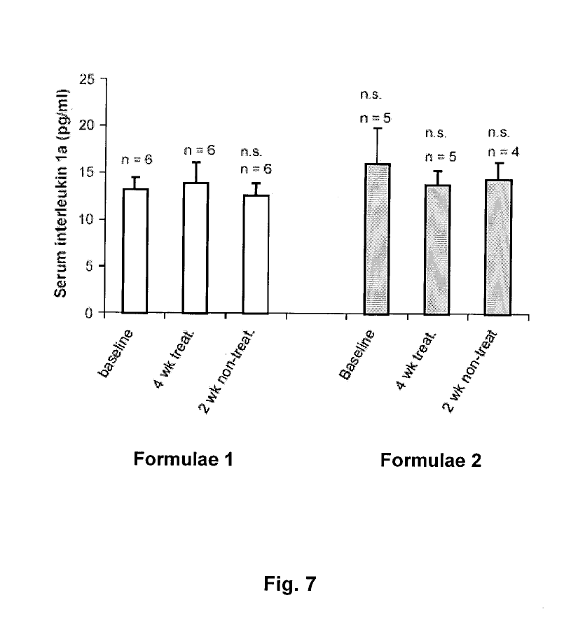

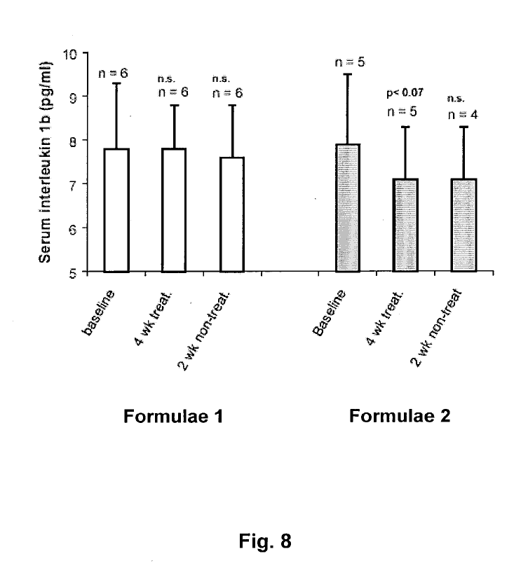

FIG. 7 shows the paired t-test analysis of the test results of interleukin 1a, determined in plasma samples as pg/ml before (baseline), after 4 weeks supplement (treatment), and after 2 weeks washout (non-treatment). FIG. 8 shows the paired t-test analysis of the test results of interleukin 1b, determined in plasma samples as pg/ml before (baseline), after 4 weeks supplement (treatment), and after 2 weeks more washout (non-treatment).

As shown, interleukin 1a was not affected by either Formulae 1 or Formulae 2 supplementations. However, interleukin 1b showed a tendency toward reduction by Formulae 2 (the data pooled from 4 weeks treatment plus 2 weeks washout gave p<0.05, FIG. 8), but not with Formulae 1. It is known that the serum level of pro-inflammatory cytokines are strong indicators of endogenous oxidative stress, because activated phagocytic cells initiate an inflammatory response by producing both oxygen radicals and inflammatory cytokines. The serum levels of interleukins 1a and 1b can be used as indicators of oxidative stress leading to DNA damage. The data shown in FIG. 8 supported the results obtained with the (8-OH) adducts and plasma/serum thiol biomarkers, which showed that endpoints sensitive to endogenous oxidative stress were modulated toward reduced DNA damage potential in vivo by the instant nutritional supplement compositions, especially by the C-Med-100® containing Formulae 2.

As illustrated by the results of this study, the two instant anti-aging supplement compositions significantly reduced DNA and pro-oxidant induced damage when estimated by several independently regulated biomarkers. These data show the feasibility of preventing humans from accumulating DNA damage and the aging consequences thereof, by administering the broad spectrum anti-aging compositions of Formulae 1 and Formulae 2 for four weeks.

More importantly, although the nutritional supplement composition of Formulae 1 provided anti-aging properties, the addition of C-MED-100® in the multi-component composition of Formulae 1 further enhanced the anti-aging properties of the composition. While the reason for this enhanced efficacy is not known, it is likely related to C-Med-100®'s known DNA repair enhancing property, presumably via NF-kB inhibition, and the synergetic effect obtained from the combination of C-MED-100® and other nutritional supplements described in the embodiment of the present invention. With the combination of the instant multi-component nutritional supplement with C-MED-100®, the present invention provides an effective anti-aging treatment means by decreasing DNA damage, increasing DNA repair, and improving immune function of human body.

Below shows the second example of the anti-aging nutritional supplement compositions of the present invention, which includes a further functional group, an inflammatory process support.

The second example of the anti-aging nutritional supplement compositions are as follows:

A morning composition comprises in four caplets:

(1) Vitamins

Vitamin A (as retinyl palmitate and 85% as beta-carotene from D. salina algae) 3,600 IU

Vitamin C (as ascorbyl palmitate and ascorbic acid) 200 mg

Vitamin D (as cholecalciferol) 80 IU

Vitamin E (as d-alpha tocopheryl succinate and with mixed tocopherols) 100 IU

Vitamin K (as phytonadione) 150 mcg

Thiamin (as thiamin HCl) 10 mg

Riboflavin (as riboflavin and riboflavin 5-phosphate) 8 mg

Niacin (as niacinamide and niacin) 140 mg

Vitamin B6 (as pyridoxine HCl and pyridoxal 5-phosphate) 24 mg

Folate (as folic acid) 100 mcg

Vitamin B12 (as cyanocobalamin) 160 mcg

Biotin 100 mcg

Pantothenic acid (as D-calcium pantothenate) 24 mg

(2) Minerals

Calcium (as calcium carbonate and calcium citrate) 600 mg

Iodine (as potassium iodide and from kelp) 60 mcg

Zinc (as zinc glycinate) 4 mg

Selenium (as selenomethionine) 60 mcg

Copper (as copper lysinate) 0.4 mg

Manganese (as manganese gluconate) 0.4 mg

Chromium (as chronium polyicotinate) 100 mcg

Molybdenum (as sodium molybdate) 20 mcg

(3) Inflammatory Process Support—Blend 100 mg

Turmeric rhizome extract (95% curcuminoids), Quercetin dehydrate and Cayenne pepper (fruit)

(4) Blood Sugar/Insulin Support—Blend

Vanadium (as vanadyl sulfate) 50 mcg

Fenugreek seed, Alpha-lipoic acid and Coenzyme Q-10 80 mg

(5) Botanical Antioxidants

Green tea leaf extract (40% catechin and polyphenols) 100 mg

Anthocyanins (from bilberry fruit and grape skin extracts) 10 mg

Ginkgo biloba leaf extract (24% ginkgo flavonglycosides, 6% sesquiterpene lactones) 100 mg

Guarana seed extract (16 mg of naturally occuring caffeine) 80 mg

(6) Methylating Factors

Betaine HCl 8 mg

Sulfur (from MSM—methylsulfonylmethane) 2.5 mg

(7) DNA Repairer

C-Med-100® (extract of Uncaria tomentosa, standardized to 8% Carboxy alkyl esters manufactured by Laboratorio Centroflora (Sao Paulo, Brazil) 175 mg

(8) Fat Metabolizers—Blend 50 mg

Gotu kola leaf, L-Carnitine-L-tartrate and Acetyl L-carnitine HCl

(9) Absorption Enhancers

Phosphatidylcholine (from soy lecithin) 50 mg

(10) Brain Function Support

dimethylaminoethanol (DMAE) bitartrate 50 mg

(11) Whole Foods—Blend 300 mg

Blue-green algae, Spirulina algae and Green barley grass (aerial parts)

(12) Cellular Energizers

Cordyceps sinensis fungus extract (1% cordycepic acid) 25 mg

Royal Jelly 3× (5% 10-HDA) 20 mg

(13) Nucleotides-Precursors for Gene Expression

Chlorella algae (10% RNA—ribonucleic acid) 50 mg

(14) Amino Acids—Blend 275 mg

L-Glutamine, L-Phenylalanine, L-Tyrosine, Taurine and N-Acetyl-L-cysteine

(15) Fatty Acid Complex 400 mg

Soybean lecithin (linoleic acid (29.5%), alpha-linolenic acid (3.5%), oleic acid (4.5%), borage seed oil (10% gamma-linolenic acid), evening primrose oil (4.8% GLA), fish body oil (4.5% eicosapentaenoic acid, 3.0% docosahexaenoic acid)

(16) Digestive Enzymes—Blend 1,760 units

Amylase, Neutral protease, Lactase, Lipase and Cellulase

The morning composition further comprises microcrystalline cellulose, croscarmellose sodium, stearic acid, calcium silicate, magnesium stearate, silica, and film coat (hydroxypropyl methylcellulose, hydroxypropyl cellulose and polyethylene glycol).

A midday composition comprises in four caplets:

(1) Vitamins

Vitamin A (as retinyl palmitate and 85% as beta-carotene from D. salina algae) 2,400 IU

Vitamin C (as ascorbic acid and ascorbyl palmitate) 160 mg

Vitamin D (as cholecalciferol) 40 IU

Vitamin E (as d-alpha tocopheryl succinate and with mixed tocopherols) 65 IU

Vitamin K (as phytonadione) 150 mcg

Thiamin (as thiamin HCl) 12 mg

Riboflavin (as riboflavin and riboflavin 5-phosphate) 1 mg

Niacin (as niacinamide and niacin) 140 mg

Vitamin B6 (as pyridoxine HCl and pyridoxal 5-phosphate) 4 mg

Folate (as folic acid) 65 mcg

Vitamin B12 (as cyanocobalamin) 200 mcg

Biotin 65 mcg

Pantothenic acid (as Dalcium pantothenate) 32 mg

(2) Minerals

Calcium (as calcium citrate) 200 mg

Iodine (as potassium iodide and from kelp) 15 mcg

Zinc (as zinc glycinate) 2.5 mg

Selenium (as selenomethionine) 40 mcg

Copper (as copper lysinate) 0.2 mg

Manganese (as manganese gluconate) 0.2 mg

Chromium (as chronium polyicotinate) 40 mcg

Molybdenum (as sodium molybdate) 12 mcg

(3) Inflammatory Process Support—Blend 100 mg

Turmeric rhizome extract (95% curcuminoids), Quercetin dehydrate and Cayenne pepper (fruit)

(4) Blood Sugar/insulin Support—Blend

Vanadium (as vanadyl sulfate) 32 mcg

Fenugreek seed, Alpha-lipoic acid and Coenzyme Q-10 55 mg

(5) Botanical Antioxidants

Ginkgo biloba leaf extract (24% ginkgo flavonglycosides, 6% sesquiterpene lactones) 100 mg

Guarana seed extract (16 mg of naturally occuring caffeine) 80 mg Ascorbigen 8 mg

(6) Methylating Factors

Betaine HCl 6.4 mg

Sulfur (from MSM—methylsulfonylmethane) 1.5 mg

(7) Fat Metabolizers—Blend 400 mg

L-Carnitine-L-tartrate and Acetyl L-carnitine HCl

(8) Absorption Enhancers

Phosphatidylcholine (from soy lecithin) 50 mg

(9) Brain Function Support

5-HTP (5-hydroxytryptophan) 50 mg

(10) Whole Foods—Blend 150 mg

Blue-green algae, Spirulina algae and Green barley grass (aerial parts)

(11) Cellular Energizers

Cordyceps sinensis fungus extract (1% cordycepic acid) 20 mg Royal Jelly 3× (5% 10-HDA) 12 mg

(12) Nucleotides-Precursors for Gene Expression

Chlorella algae (10% RNA—ribonucleic acid) 50 mg

(13) Amino Acids—Blend 225 mg

L-Glutamine, Taurine, L-Phenylalanine, L-Tyrosine, and N-Acetyl-L-cysteine

(14) Fatty Acid Complex 400 mg

Soybean lecithin (linoleic acid (29.5%), alpha-linolenic acid (3.5%), oleic acid (4.5%)), borage seed oil (10% gamma-linolenic acid), evening primrose oil (4.8% GLA), and fish body oil (4.5% eicosapentaenoic acid, 3.0% docosahexaenoic acid)

(15) Digestive Enzymes—Blend 1,408 units

Amylase, Neutral protease, Lactase, Lipase and Cellulase.

The midday formula further comprises Lactose, microcrystalline cellulose, croscarmellose sodium, stearic acid, calcium silicate, magnesium stearate, silica, and film coat (hydroxypropyl methylcellulose, hydroxypropyl cellulose, and polyethylene glycol).

A night composition comprises in four caplets:

(1) Vitamins

Vitamin A (as retinyl palmitate and 85% as beta-carotene from D. salina algae) 2,800 IU

Vitamin C (as ascorbic acid and ascorbyl palmitate) 400 mg

Vitamin D (as cholecalciferol) 60 IU

Vitamin E (as d-alpha tocopheryl succinate and with mixed tocopherols) 80 IU

Vitamin K (as phytonadione) 150 mcg

Thiamin (as thiamin HCl) 5 mg

Riboflavin (as riboflavin and riboflavin 5-phosphate) 10 mg

Niacin (as niacinamide and niacin) 140 mg

Vitamin B6 (as pyridoxine HCl and pyridoxal 5-phosphate) 15 mg

Folate (as folic acid) 160 mcg

Vitamin B12 (as cyanocobalamin) 240 mcg

Biotin 80 mcg

Pantothenic acid (as D-calcium pantothenate) 40 mg

(2) Minerals

Calcium (as dicalcium phosphate) 215 mg

Iodine (as potassium iodide and from kelp) 24 mcg

Magnesium (as magnesium oxide and magnesium glycinate) 265 mg

Zinc (as zinc glycinate) 3 mg

Selenium (as selenomethionine) 48 mcg

Copper (as copper lysinate) 0.2 mg

Manganese (as manganese gluconate) 0.2 mg

Chromium (as chronium polyicotinate) 80 mcg

Molybdenum (as sodium molybdate) 16 mcg

(3) Inflammatory Process Support—Blend 100 mg

Turmeric rhizome extract (95% curcuminoids), Quercetin dehydrate and Cayenne pepper (fruit)

(4) Blood Sugar/Linsulin Support—Blend

Vanadium (as vanadyl sulfate) 40 mcg

Fenugreek seed, Alpha-lipoic acid and Coenzyme Q-10 67 mg

(5) Botanical Antioxidants—Blend 147 mg

Cruciferous vegetable concentrate: broccoli, kale, radish (2% glucosinolates), Grape skin extract (37% total polyphenols), Tomato lycopene extract (20% lycopene), Rosemary 4:1 extract (aerial parts), Pycnogenol (pine tree bark extract) and Lutein (from marigold flower extract)

(6) Methylating Factors

Betaine (as betaine-HCl) 5 mg

Sulfur (from MSM—methylsulfonylmethane) 2 mg

(7) DNA Repair Agent

C-Med-100® (extract of Uncaria tomentosa, standardized to 8% Carboxy alkyl esters manufactured by Laboratorio Centroflora (Sao Paulo, Brazil) 175 mg

(8) Fat Metabolizers—Blend 30 mg

L-Camitine-L-tartrate and Acetyl L-carnitine HCl

(9) Absorption Enhancers

Phosphatidylcholine (from soy lecithin) 40 mg

(10) Brain Function Support—Blend 161 mg

Inositol, Valerian root and Melatonin

(11) Whole Foods—Blend 140 mg

Blue-green algae, Spirulina algae and Green barley grass (aerial parts)

(12) Cellular Energizers

Cordyceps sinensis fungus extract (1% cordycepic acid) 16.5 mg

Royal Jelly 3× (5% 10-HDA) 18 mg

(13) Nucleotides-Precursors for Gene Expression

Chlorella algae (10% RNA—ribonucleic acid) 50 mg

(14) Amino Acids—Blend 1,148 mg

L-Glutamine, L-Arginine (as L-arginine HCl), L-Ornithine (as L-ornithine HCl), L-Tyrosine, Taurine and N-Acetyl-L-cysteine

(15) Fatty Acid Complex 400 mg

Soybean lecithin (linoleic acid (29.5%), alpha-linolenic acid (3.5%), oleic acid (4.5%)), borage seed oil (10% gamma-linolenic acid), evening primrose oil (4.8% GLA), and fish body oil (4.5% eicosapentaenoic acid, and 3.0% docosahexaenoic acid)

(16) Probiotic Complex 100 million CFU

Lactobacillus acidophilus, Lactobacillus plantarum, Bifidobacterium bifidum and Lactobacillus casei

(17) Digestive Enzymes—Blend 1,169 Units

Amylase, Neutral protease, Lactase, and Lipase and Cellulase

The night composition further comprises: microcrystalline cellulose, croscarmellose sodium, stearic acid, calcium silicate, magnesium stearate, silica, and film coat (hydroxypropyl methylcellulose, hydroxypropyl cellulose, and polyethylene glycol).

As illustrated, several functional groups, such as botanical antioxidant, fat metabolizer, brain function support, amino acids, and particularly fatty acid complex, are further enhanced in the compositions of the second example. Moreover, chorella algae is used to provide RNA instead of yeast, which has less allergic response in comparison to yeast. Additionally, valerian root and melatonin are used to substitute Kava Kava root extract in the composition as the brain function support.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the claims appended herewith.

What is claimed is:

1. An anti-aging nutritional supplement composition, comprising:
(a) vitamins,
(b) minerals,
(c) a blood sugar/insulin support,
(d) botanical antioxidants,
(e) a methylating factor,
(f) a DNA repair agent,
(g) a fat metabolizer,
(h) an absorption enhancer,
(i) a brain function support,
(j) a cellular energizer,
(k) a nucleotide precursor,
(l) amino acids,
(m) a fatty acid complex,
(n) a probiotic complex, and
(o) digestive enzymes.

2. The anti-aging nutritional supplement composition of claim 1 further comprising a pharmaceutical carrier.

3. The anti-aging nutritional supplement composition of claim 1 further comprising an inflammatory process support.

4. The anti-aging nutritional supplement composition of claim 3, wherein said inflammatory process support is a blend of turmeric rhizome extract (95% curcuminoids), quercetin dehydrate and cayenne pepper (fruit).

5. The anti-aging nutritional supplement composition of claim 1 further comprising whole foods.

6. The anti-aging nutritional supplement composition of claim 5, wherein said whole foods are a blend blue-green algae, spirulina algae and green barley grass (aerial parts).

7. The anti-aging nutritional supplement composition of claim 1, wherein said vitamins comprise vitamin A (as retinyl palmitate and 85% as beta-carotene from D. salina algae), vitamin C (as ascorbyl palmitate and ascorbic acid), vitamin D (as cholecalciferol), vitamin E (as d-alpha tocopheryl succinate and with mixed tocopherols), vitamin K (as phytonadione), thiamin (as thiamin HCl), riboflavin (as riboflavin and riboflavin 5-phosphate), niacin (as niacinamide and niacin), vitamin B6 (as pyridoxine HCl and pyridoxal 5-phosphate), folate (as folic acid), vitamin B12 (as cyanocobalamin), biotin, and pantothenic acid (as D-calcium pantothenate).

8. The anti-aging nutritional supplement composition of claim 1, wherein said minerals comprise calcium (as calcium carbonate and calcium citrate), iodine (as potassium iodide and from kelp), zinc (as zinc glycinate), selenium (as selenomethionine), copper (as copper lysinate), manganese (as manganese gluconate), chromium (as chronium polyicotinate), and molybdenum (as sodium molybdate).

9. The anti-aging nutritional supplement composition of claim 8, wherein said minerals further comprise magnesium (as magnesium oxide and magnesium glycinate).

10. The anti-aging nutritional supplement composition of claim 1, wherein said blood sugar/insulin support is a blend of vanadium (as vanadyl sulfate), fenugreek seed, alpha-lipoic acid and coenzyme Q-10.

11. The anti-aging nutritional supplement composition of claim 1, wherein said botanical antioxidants are a mixture of green tea leaf extract (40% catechin and polyphenols), anthocyanins (from bilberry fruit and grape skin extracts), ginkgo biloba leaf extract (24% ginkgo flavonglycosides, 6% sesquiterpene lactones), guarana seed extract (naturally occuring caffeine).

12. The anti-aging nutritional supplement composition of claim 1, wherein said botanical antioxidants are a mixture of ginkgo biloba leaf extract (24% ginkgo flavonglycosides, 6% sesquiterpene lactones), guarana seed extract (naturally occurring caffeine), and ascorbigen.

13. The anti-aging nutritional supplement composition of claim 1, wherein said botanical antioxidants are a mixture of cruciferous vegetable concentrate including broccoli, kale and radish (2% glucosinolates); grape skin extract (37% total polyphenols), tomato lycopene extract (20% lycopene), rosemary extract (aerial parts), pycnogenol (pine tree bark extract) and lutein (from marigold flower extract).

14. The anti-aging nutritional supplement composition of claim 1, wherein said methylating factor comprise betaine HCl and sulfur (from methylsulfonylmethane).

15. The anti-aging nutritional supplement composition of claim 1, wherein said DNA repair agent is an extract of Uncaria tomentosa standardized to 8% carboxy alkyl esters.

16. The anti-aging nutritional supplement composition of claim 1, wherein said fat metabolizer comprises L-carnitine-L-tartrate and acetyl L-carnitine HCl.

17. The anti-aging nutritional supplement composition of claim 1, wherein said fat metabolizer further comprises gotu kola leaf.

18. The anti-aging nutritional supplement composition of claim 1, wherein said absorption enhancer is phosphatidylcholine (from soy lecithin).

19. The anti-aging nutritional supplement composition of claim 1, wherein said brain function support is dimethylaminoethanol bitartrate.

20. The anti-aging nutritional supplement composition of claim 1, wherein said brain function support is 5-hydroxytryptophan.

21. The anti-aging nutritional supplement composition of claim 1, wherein said brain function support is a mixture of inositol, valerian root and melatonin.

22. The anti-aging nutritional supplement composition of claim 1, wherein said cellular energizer comprises cordyceps sinensis fungus extract (1% cordycepic acid) and royal jelly 3× (5% 10-HDA).

23. The anti-aging nutritional supplement composition of claim 1, wherein said nucleotides precursors is chlorella algae (10% ribonucleic acid).

24. The anti-aging nutritional supplement composition of claim 1, wherein said nucleotides precursors is yeast (10% ribonucleic acid).

25. The anti-aging nutritional supplement composition of claim 1, wherein said amino acids are a mixture of L-glutamine, L-phenylalanine, L-tyrosine, taurine and N-acetyl-L-cysteine.

26. The anti-aging nutritional supplement composition of claim 1, wherein said amino acids are a mixture of L-glutamine, L-arginine (as L-arginine HCl), L-ornithine (as L-ornithine HCl), L-tyrosine, taurine and N-acetyl-L-cysteine.

27. The anti-aging nutritional supplement composition of claim 1, wherein fatty acid complex comprise soybean lecithin (linoleic acid (29.5%), alpha-linolenic acid (3.5%), oleic acid (4.5%)), borage seed oil (10% gamma-linolenic acid), evening primrose oil (4.8% GLA), and fish body oil (4.5% eicosapentaenoic acid and 3.0% docosahexaenoic acid).

28. The anti-aging nutritional supplement composition of claim 1, wherein digestive enzymes are a blend of amylase, neutral protease, lactase, lipase and cellulase.

29. The anti-aging nutritional supplement composition of claim 1 wherein said probiotic complex comprises lactobacillus acidophilus, lactobacillus plantarum, bifidobacterium bifidum, and lactobacillus casei.

30. An anti-aging nutritional supplement system comprising:
(i) a first nutritional supplement composition to be administered in the morning, said first composition comprising:
(a) vitamins, including:
vitamin A (as retinyl palmitate and 85% as beta-carotene from D. salina algae), 3,600 IU
vitamin C, (as ascorbyl palmitate and ascorbic acid) 200 mg
vitamin D, (as cholecalciferol) 80 IU
vitamin E (as d-alpha tocopheryl succinate and with mixed tocopherols), 100 IU
vitamin K (as phytonadione), 150 mcg
thiamin (as thiamin HCl), 10 mg
riboflavin (as riboflavin and riboflavin 5-phosphate), 8 mg
niacin (as niacinamide and niacin), 140 mg
vitamin B6 (as pyridoxine HCl and pyridoxal 5-phosphate), 24 mg
folate (as folic acid), 100 mcg
vitamin B12 (as cyanocobalamin), 160 mcg
biotin, 100 mcg
pantothenic acid (as D-calcium pantothenate), 24 mg
(b) minerals, including:
calcium (as calcium carbonate and calcium citrate), 600 mg
iodine (as potassium iodide and from kelp), 60 mcg
zinc (as zinc glycinate), 4 mg
selenium (as selenomethionine), 60 mcg
copper (as copper lysinate), 0.4 mg
manganese (as manganese gluconate), 0.4 mg
chromium (as chronium polyicotinate), 100 mcg
molybdenum (as sodium molybdate), 20 mcg
(c) an inflammatory process support, including a blend of 100 mg of:
turmeric rhizome extract (95% curcuminoids), quercetin dehydrate and cayenne pepper (fruit),
(d) a blood sugar/insulin support, including a blend of:
vanadium (as vanadyl sulfate), 50 mcg
fenugreek seed, alpha-lipoic acid, and coenzyme Q-10, 80 mg
(e) botanical antioxidants, including:
green tea leaf extract (40% catechin and polyphenols), 100 mg
anthocyanins (from bilberry fruit and grape skin extracts), 10 mg
ginkgo biloba leaf extract (24% ginkgo flavonglycosides, and 6% sesquiterpene lactones), 100 mg
guarana seed extract (16 mg of naturally occurring caffeine), 80 mg
(f) methylating factors, including:
betaine HCl, 8 mg
sulfur (from methylsulfonylmethane), 2.5 mg
(g) a DNA repair agent, including:
extract of Uncaria tomentosa, standardized to 8% carboxy alkyl esters, 175 mg
(h) fat metabolizers, including a blend of 50 mg of:
gotu kola leaf, L-carnitine-L-tartrate and acetyl L-carnitine HCl,
(i) an absorption enhancer including:
phosphatidylcholine (from soy lecithin), 50 mg
(j) a brain function support, including:
DMAE bitartrate, 50 mg
(k) whole foods, including a blend of 300 mg of:
blue-green algae, spirulina algae and green barley grass (aerial parts),
(l) cellular energizers, including:
cordyceps sinensis fungus extract (1% cordycepic acid), 25 mg
royal jelly 3× (5% 10-HDA), 20 mg
(m) nucleotides-precursors for gene expression, including:
chlorella algae (10% RNA—ribonucleic acid), 50 mg
(n) amino acids, including a blend of 275 mg of:
L-glutamine, L-phenylalanine, L-tyrosine, taurine, and N-acetyl-L-cysteine,
(o) a fatty acid complex, including 400 mg of:
soybean lecithin (linoleic acid (29.5%), alpha-linolenic acid (3.5%), oleic acid (4.5%)), borage seed oil (10% gamma-linolenic acid), evening primrose oil (4.8% GLA), and fish body oil (4.5% eicosapentaenoic acid, and 3.0% docosahexaenoic acid), and
(p) digestive enzymes, including a blend of 1,760 units of:
amylase, neutral protease, lactase, lipase and cellulase
(ii) a second nutritional supplement composition to be administered at midday, said second composition comprising:
(a) vitamins, including:
vitamin A (as retinyl palmitate and 85% as beta-carotene from D. salina algae), 2,400 IU
vitamin C (as ascorbic acid and ascorbyl palmitate), 160 mg
vitamin D (as cholecalciferol), 40 IU
vitamin E (as d-alpha tocopheryl succinate and with mixed tocopherols), 65 IU
vitamin K (as phytonadione), 150 mcg
thiamin (as thiamin HCl), 12 mg
riboflavin (as riboflavin and riboflavin 5-phosphate), 1 mg
niacin (as niacinamide and niacin), 140 mg
vitamin B6 (as pyridoxine HCl and pyridoxal 5-phosphate), 4 mg
folate (as folic acid), 65 mcg
vitamin B12 (as cyanocobalamin), 200 mcg
biotin, 65 mcg
pantothenic acid (as D-calcium pantothenate), 32 mg
(b) minerals, including:
calcium (as calcium citrate), 200 mg
iodine (as potassium iodide and from kelp), 15 mcg
zinc (as zinc glycinate), 2.5 mg
selenium (as selenomethionine), 40 mcg
copper (as copper lysinate), 0.2 mg
manganese (as manganese gluconate), 0.2 mg
chromium (as chronium polyicotinate), 40 mcg
molybdenum (as sodium molybdate), 12 mcg
(c) an inflammatory process support, including a blend of 100 mg of:
turmeric rhizome extract (95% curcuminoids), quercetin dehydrate and cayenne pepper (fruit),
(d) a blood sugar/insulin support, including a blend of:
vanadium (as vanadyl sulfate), 32 mcg
fenugreek seed, alpha-lipoic acid, and coenzyme Q-10, 55 mg
(e) botanical antioxidants, including:
ginkgo biloba leaf extract (24% ginkgo flavonglycosides, and 6% sesquiterpene lactones), 100 mg
guarana seed extract (16 mg of naturally occurring caffeine), 80 mg ascorbigen, 8 mg
(f) a methylating factor, including:
betaine HCl, 6.4 mg
sulfur (from methylsulfonylmethane), 1.5 mg
(g) fat metabolizers, including a blend of 400 mg of:
L-carnitine-L-tartrate, and acetyl L-carnitine HCl,
(h) an absorption enhancer, including:
phosphatidylcholine (from soy lecithin), 50 mg
(i) a brain function support, including:
5-HTP (5-hydroxytryptophan), 50 mg
(j) whole foods, including a blend of 150 mg of:
blue-green algae, spirulina algae and green barley grass (aerial parts),
(k) cellular energizers, including:
cordyceps sinensis fungus extract (1% cordycepic acid), 20 mg royal jelly 3× (5% 10-HDA), 12 mg
(l) nucleotides-precursors for gene expression, including:
chlorella algae (10% RNA—ribonucleic acid), 50 mg
(m) amino acids, including a blend of 225 mg of:
L-glutamine, taurine, L-phenylalanine, L-tyrosine, and N-acetyl-L-cysteine,
(n) a fatty acid complex, including 400 mg of:
soybean lecithin (linoleic acid (29.5%), alpha-linolenic acid (3.5%), oleic acid (4.5%)), borage seed oil (10% gamma-linolenic acid), evening primrose oil (4.8% GLA), and fish body oil (4.5% eicosapentaenoic acid, and 3.0% docosahexaenoic acid), and
(o) digestive enzymes, including a blend of 1,408 units of:
amylase, neutral protease, lactase, lipase and cellulose; and
(iii) a third nutritional supplement composition to be administered in the night, said third composition comprising:
(a) vitamins, including:
vitamin A (as retinyl palmitate and 85% as beta-carotene from D. salina algae), 2,800 IU
vitamin C (as ascorbic acid and ascorbyl palmitate), 400 mg
vitamin D (as cholecalciferol), 60 IU
vitamin E (as d-alpha tocopheryl succinate and with mixed tocopherols), 80 IU
vitamin K (as phytonadione), 150 mcg
thiamin (as thiamin HCl), 5 mg
riboflavin (as riboflavin and riboflavin 5-phosphate), 10 mg
niacin (as niacinamide and niacin), 140 mg
vitamin B6 (as pyridoxine HCl and pyridoxal 5-phosphate), 15 mg
folate (as folic acid), 160 mcg
vitamin B12 (as cyanocobalamin), 240 mcg
biotin, 80 mcg
pantothenic acid (as D-calcium pantothenate), 40 mg
(b) minerals, including:
calcium (as dicalcium phosphate), 215 mg
iodine (as potassium iodide and from kelp), 24 mcg
magnesium (as magnesium oxide and magnesium glycinate), 265 mg
zinc (as zinc glycinate), 3 mg
selenium (as selenomethionine), 48 mcg
copper (as copper lysinate), 0.2 mg
manganese (as manganese gluconate), 0.2 mg
chromium (as chronium polyicotinate), 80 mcg
molybdenum (as sodium molybdate), 16 mcg
(c) an inflammatory process support, including a blend of 100 mg of:
turmeric rhizome extract (95% curcuminoids), quercetin dehydrate and cayenne pepper (fruit),
(d) a blood sugar/insulin support, including a blend of:
vanadium (as vanadyl sulfate), 40 mcg
fenugreek seed, alpha-lipoic acid and coenzyme Q-10, 67 mg
(e) botanical antioxidants, including a blend of 147 mg of:
cruciferous vegetable concentrate: broccoli, kale, radish (2% glucosinolates), grape skin extract (37% total polyphenols), tomato lycopene extract (20% lycopene), rosemary 4:1 extract (aerial parts), pycnogenol (pine tree bark extract) and lutein (from marigold flower extract),
(f) methylating factors, including:
betaine (as betaine-HCl), 5 mg
sulfur (from methylsulfonylmethane), 2 mg
(g) a DNA repair agent, including:
extract of uncaria tomentosa, standardized to 8% carboxy alkyl esters, 175 mg
(h) fat metabolizers, including a blend of 30 mg of:
L-carnitine-L-tartratef and acetyl L-carnitine HCl,
(i) an absorption enhancer, including:
phosphatidylcholine (from soy lecithin), 40 mg
(j) a brain function support, including a blend of 161 mg of:
inositol, valerian root, and melatonin,
(k) whole foods, including a blend of 140 mg of:
blue-green algae, spirulina algae, and green barley grass (aerial parts),
(l) cellular energizers, including:
cordyceps sinensis fungus extract (1% cordycepic acid), 16.5 mg
royal jelly 3× (5% 10-HDA), 18 mg
(m) nucleotides-precursors for gene expression, including:
chlorella algae (10% RNA—ribonucleic acid), 50 mg
(n) amino acids, including a blend of 1,148 mg of:
L-glutamine, L-arginine (as L-arginine HCl), L-ornithine (as L-omithine HCl), L-tyrosine, taurine, and N-acetyl-L-cysteine,
(o) a fatty acid complex of 400 mg of:
soybean lecithin (linoleic acid (29.5%), alpha-linolenic acid (3.5%), oleic acid (4.5%)), borage seed oil (10% gamma-linolenic acid), evening primrose oil (4.8% GLA), and fish body oil (4.5% eicosapentaenoic acid, and 3.0% docosahexaenoic acid),
(p) a probiotic complex, including 100 million CFU of:
lactobacillus acidophilus, lactobacillus plantarum, bifidobacterium bifidum and lactobacillus casei, and
(q) digestive enzymes, including a blend of 1,169 units of:
amylase, neutral protease, lactase, and lipase and cellulose.

31. The anti-aging nutritional supplement system of claim 30, wherein said first nutritional supplement composition further comprises microcrystalline cellulose, croscarmellose sodium, stearic acid, calcium silicate, magnesium stearate, silica, and film coat (hydroxypropyl methylcellulose, hydroxypropyl cellulose and polyethylene glycol).

32. The anti-aging nutritional supplement system of claim 30, wherein said second nutritional supplement composition further comprises Lactose, microcrystalline cellulose, croscarmellose sodium, stearic acid, calcium silicate, magnesium stearate, silica, and film coat (hydroxypropyl methylcellulose, hydroxypropyl cellulose, and polyethylene glycol).

33. The anti-aging nutritional supplement system of claim 30, wherein said third nutritional supplement composition further comprises microcrystalline cellulose, croscarmellose sodium, stearic acid, calcium silicate, magnesium stearate, silica, and film coat (hydroxypropyl methylcellulose, hydroxypropyl cellulose, and polyethylene glycol).

34. A method of anti-aging treatment comprising orally administering an effective amount of an anti-aging nutritional supplement composition daily, wherein said anti-aging supplement composition comprises:
(a) vitamins,
(b) minerals,
(c) an inflammatory process support,
(d) a blood sugar/insulin support,
(e) botanical antioxidants,
(f) a methylating factor,
(g) a DNA repair agent,
(h) a fat metabolizer,
(i) an absorption enhancer,
(j) a brain function support,
(k) a cellular energizer,
(l) a nucleotide precursor,
(m) amino acids,
(n) a fatty acid complex,
(o) a probiotic complex, and
(p) digestive enzymes.

35. The method of anti-aging treatment of claim 34, wherein said anti-aging nutritional supplement composition further comprises a pharmaceutical carrier.

36. The method of anti-aging treatment of claim 34, wherein said anti-aging nutritional supplement composition further comprises whole foods.

37. The method of anti-aging treatment of claim 34, wherein said treatment comprising orally administering said anti-aging supplement composition three doses daily.

38. The method of anti-aging treatment of claim 37, wherein said three doses are administered once in the morning, once at midday and once at night.

39. The method of anti-aging treatment of claim 38, wherein individual components and quantities of said individual components of said composition can be different among said three doses for supporting different needs according to human body's bio-cycle.
